(19) United States
(12) Patent Application Publication
(10) Pub. No.: US 2004/0191295 A1
(43) Pub. Date: Sep. 30, 2004

(54) Composition and method for viral inhibition

(76) Inventors: Mary Frances Locniskar, Lawrence, KS (US); Bryon Walter Petschow, Newburgh, IN (US); Robert Alexander Burns, Evansville, IN (US); Virginia Sue Sickles, Evansville, IN (US); James Wayne Hansen, Evansville, IN (US); Steven Charles Rumsey, Evansville, IN (US)

Correspondence Address: Robert S. Thomas, Nelson Mullins Riley & Scarborough LLP, Keenan Building, Third Floor, 1330 Lady Street, Columbia, SC, 29201, US

(21) Appl. No.: 10/401,390

(22) Filed: Mar. 28, 2003

(51) Int. Cl. A61K 31/7012; A61K 47/00

(52) U.S. Cl. 424/439; 514/53

(57) ABSTRACT

A method for inhibiting Rotavirus infection by administering effective amount of lactulose is provided. Lactulose can be administered alone, in combination with other nutrient supplements such as vitamins, or as part of a nutritional product such as infant formula.

### BACKGROUND OF THE INVENTION

1. Field of the Invention

The present invention relates generally to methods for inhibiting viral infections and more particularly to the use of oligosaccharide-containing compositions for inhibiting the infection of mammalian cells by Rotavirus.

1. Description of the Related Art

Worldwide, Rotavirus infection is the most frequent cause of diarrhea in infants, toddlers and children. The illness can be as severe as cholera, resulting in severe dehydration and death. Globally, Rotavirus accounts for an estimated 125 million cases of diarrhea each year and represents between 30 to 40% of all hospitalizations for diarrhea in children under five years of age. In the U.S., about 1 in 78 children are hospitalized with Rotavirus-caused diarrhea by the time they reach the age of five. In developing countries, between 600,000 to 800,000 children die from Rotavirus infections every year. This accounts for about 5% of all deaths among children under five.

Conventional medicine has relied on the development of a vaccine to prevent Rotavirus-induced diarrhea. Thus, developing a vaccine against Rotavirus has become a major public health goal, but one that has proven elusive. Although the Rotavirus genus has been sequenced and much is known about its structure, biochemistry, immunology and pathology, attempts to produce a safe vaccine have been unsuccessful to date. (J. Ped. Gastr. Nutr. 30:10-11, 2000.) In 1999, a vaccine was withdrawn from the U.S. market when it became apparent that some infants who received it developed intussusception within one to three weeks from vaccination. Intussusception is the folding of a portion of the bowel into itself. This folding strangles the tissue of the bowel walls and of the mesenteric region that contains the blood vessels that feed the bowel. Thus, there is a present need for methods that, without causing undesirable side effects, may prevent Rotavirus diarrhea or ameliorate its severity.

It has been observed that the incidence of diarrhea is reduced in breast-fed infants. Much of this protective activity was thought due to the presence in human milk of certain antibodies such as secretory IgA that are known to be effective against Rotavirus. However, the level of these antibodies present in human milk does not seem sufficient to fully account for the effective protection against Rotavirus infections. It is now believed that human milk may contain other active protective agents against Rotavirus and other pathogens.

Mammalian cells, including human cells, present certain sequences of complex carbohydrates on their surface that play a role in the cell communication with the outside world. Bacteria, viruses and other pathogens may use carbohydrate-binding proteins to attach to those carbohydrate sites on the cell surface and, thus, initiate cell infection. However, certain carbohydrates that may be present in the mucous layer that lines all epithelial cells or in body fluids in contact with the cells may act as decoys and inhibit the attachment of pathogens to cells. These decoy substances have carbohydrate structures similar to those present on the cell surface but are not attached to the target cell. The decoy compounds bind to the pathogen's carbohydrate-binding proteins. Pathogens bound to the decoys are cleared from the body by the excretory mechanism characteristic of the tissue in question, for example, mucociliary action in the respiratory tract or bulk fluid movement in the gastrointestinal tract.

The use of oligosaccharides to inhibit binding of pathogens to mammalian cells is known in the art. For example, U.S. Pat. No. 6,001,819 discloses a method of inhibiting colonization of a bacteria selected from the group consisting of Streptococcus pneumoniae, Haemophilus influenza, Haemophilus parainfluenza, Burkholderia (Pseudomonas) cepacia and mixtures thereof. The method comprises administering to the patient a binding-inhibiting effective amount of a synthetic carbohydrate.

U.S. Pat. No. 5,827,526, on the other hand, discloses a method of reducing the duration of diarrhea in a human comprising administering a certain mixture of fructooligosaccharides to the human. An effective level of nystose, 1^{F}-β-fructofuranosyl, or mixtures thereof is used to reduce the duration of diarrhea. The mixture of oligosaccharides may be administered as part of a nutritional product. This product may be an infant formula, follow-on formula, toddler's beverage, milk, yogurt or fermented product. It may also be a candy, chewing gum, a tablet, a lozenge or a liquid.

Similarly, U.S. Pat. No. 5,658,893 discloses a method of inhibiting the infection of an animal or human gastrointestinal epithelial cells by Rotavirus comprising administering to the animal or human certain sulphated polysaccharides. The specifically disclosed polysaccharides are dextran sulfate, lambda-carrageenan, iota-carrageenan, and kappa-carrageenan. The preferred form is lambda-carrageenan and it can be administered by adding it to nutritional substances.

While the inhibitory effect of oligosaccharides and polysaccharides on the binding of pathogens to mammalian cells is generally known in the art, different viruses respond differently to the various oligo- and poly-saccharides. The response of a particular virus to a particular saccharide cannot be predicted.

Lactulose is a synthetic disaccharide (4-O-β-D-galactopyranosil-D-fructofuranose) that does not occur in nature. It can be produced, in small scale, by heat-treating milk and, in a large scale, by alkaline isomerization of lactose. Lactulose has been used at high dosages, to treat certain medical conditions, namely constipation and hepatic encephalopathy. Furthermore, it is recognized as a prebiotic ingredient that effectively promotes the growth of bifidobacteria in the colon. However, lactulose is not known as having an inhibitory effect on the binding of Rotavirus to an epithelial cell line. It has now been discovered, unexpectedly, that lactulose inhibits the binding of Rotavirus to epithelial cells. Lactulose's inhibitory properties, together with the oligosaccharide's ability to be added to nutritional substances, permitted the development of an effective method to prevent or ameliorate the severity of Rotavirus-induced diarrhea in infants, toddlers and children.

### SUMMARY OF THE INVENTION

Briefly, the present invention is directed to a novel method to inhibit infection of mammalian cells by Rotavirus, the method comprising administering to the mammal an effective amount of lactulose. The method can be applied to animals and humans, including infants, toddlers and children.

The present invention is also directed to a novel infant formula composition comprising lactulose in an amount effective to inhibit infection of mammalian cells by Rotavirus, and to nutritional substances for infants, toddlers and children comprising lactulose in an amount effective to inhibit infection of mammalian cells by Rotavirus.

### BRIEF DESCRIPTION OF THE DRAWINGS

FIG. 1 is a graph showing the result of treating MA104 cells infected with Rotavirus, at different dilution levels.

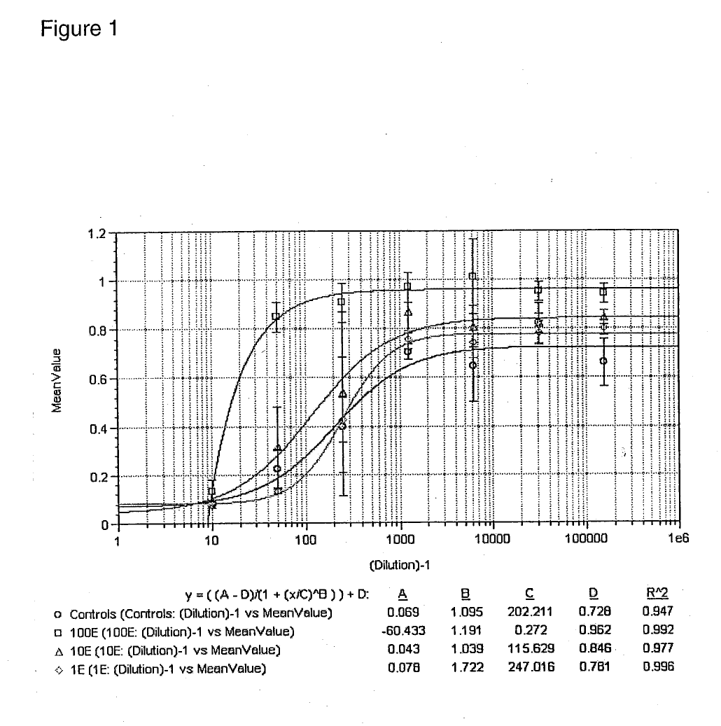

### DETAILED DESCRIPTION OF THE PREFERRED EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment.

Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

In accordance with the present invention, it has been discovered that the administration of an effective amount of lactulose to an animal or a human inhibits infection of mammalian cells by Rotavirus. This discovery permits the development of an effective method to prevent or to ameliorate the effects of Rotavirus-induced diarrhea in infants, toddlers and children.

The form of administration of lactulose in the method of the present invention is not critical, as long as an effective amount of the oligosaccharide is administered. A convenient form of administration is to add lactulose to an infant formula (including those for both term and preterm infants), follow-on formula, toddler's beverage, milk, yogurt, or fermented product. Alternatively, lactulose can be administered as a supplement not integral to formula feeding, for example, as drops, sachets or in combination with other nutrients such as vitamins. Lactulose can be also added to other nutrients suitable for feeding to young children, e.g., solid baby food, fruit juices, gelatines, cookies, candies, etc. Other examples of administering lactulose in nutrients can be developed by a person with ordinary skill in the art of infant and child nutrition. All these forms of lactulose administration, as well as others, are within the scope of the present invention.

In one embodiment of the invention, lactulose is administered as part of an infant formula. The infant formula for use in the present invention is, typically, nutritionally complete and contains suitable types and amounts of lipids, carbohydrates, proteins, vitamins and minerals. The amount of lipids or fats typically can vary from about 3 to about 7 g/100 kcal. The amount of proteins typically can vary from about 1 to about 5g/100 kcal. The amount of carbohydrates typically can vary from about 6 to about 16 g/100 kcal. Protein sources can be any used in the art, e.g., nonfat milk, whey protein, casein, soy protein, hydrolyzed protein, and amino acids. Lipid sources can be any used in the art, e.g., vegetable oils such as palm oil, soybean oil, palm olein oil, corn oil, canola oil, coconut oil, medium chain triglyceride oils, high oleic sunflower oil, and high oleic safflower oil. Carbohydrate sources can be any known in the art, e.g., lactose, glucose polymers, corn syrup solids, maltodextrins, sucrose, starch, and rice syrup solids. Conveniently, several commercially available infant formulas can be used. For example, Enfamil® Lipil with iron (available from Mead Johnson & Company, Evansville, Ind., U.S.A.) may be supplemented with an effective amount of lactulose and used to practice the method of the present invention. Particular infant formulas suitable for use in the present invention are described in Examples 3 to 8.

An effective amount for use in the present invention is typically from about 1.5 mg per kg of body weight per day to about 450mg per kg of body weight per day. In one embodiment of the invention, the amount is from about 4 mg per kg of body weight per day to about 225 mg per kg of body weight per day. In yet another embodiment the amount is from about 5 mg per kg of body weight per day to about 100 mg per kg of body weight per day.

The amount of lactulose in infant formulas for use in the present invention typically varies from about 1.5 mg/100 kcal to about 450 mg/100 kcal. In one embodiment of the present invention it varies from about 4 mg/100 kcal to about 225 mg/100 kcal, and in another embodiment from about 5 mg/100 kcal to about 100 mg/100 kcal.

The infant formula supplemented with lactulose for use in the present invention can be made using standard techniques known in the art. For example, lactulose can be added to the formula by replacing an equivalent amount of other sugars or polysaccharides. As another example, lactulose can be added to the formula in conjunction with live cultures of beneficial microorganisms such as Lactobacillus and bifidus bacteria.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### EXAMPLE 1

This example shows the results of studies conducted to evaluate the efficacy of lactulose and other oligosaccharides in inhibiting binding of Rotavirus to mammalian cells.

MA-104 cells of epithelial morphology were obtained from American Type Culture Collection and were originally derived from Rhesus monkey embryonic kidney. The cells were cultured at 37° C. in a humidified incubator and in an atmosphere of 5% CO₂, in Minimal Essential Medium with Earle's balanced salt solution (2 mM glutamine, 1 mM sodium pyruvate, 0.1 mM non-essential amino acids and 1.5 g/L sodium bicarbonate) supplemented with 10% fetal bovine serum from Hyclone, Logan, UT. The cells were passaged twice a week at a split ratio of 1:4.

Human Rotavirus strain Wa was obtained from the American Type Culture Collection. A virus stock was diluted 1:5 in media, aliquoted to 0.2 mL per vial and stored at approximately −80° C. The human Rotavirus strain, Wa (TC adapted) (Catalog No. VR-2018), originally isolated from a diarrheic stool from a human patient (Wyatt et al., Science 207: 189-191, 1980; U.S. Pat. 4,341,870), is Serotype 1 (G1) for VP7.

MTT Viability Assays were conducted in a Promega CellTiter 96® Non-Radioactive Cell Kit (Cat # G400) obtained from Promega, Madison, Wis. and used according to manufacturer's instructions.

Test compounds were prepared by dissolving each compound at 20-μg/mL final concentration in incomplete MA-104 media (Minimal Essential Medium with Earle's balanced salt solution). The compounds were sterilized by filtration through a 0.45 -micron filter. The filter-sterilized stock was stored frozen at less than −20° C.

Virus Inhibition Assay: Virus strain Wa (1:2 dilution from stock) was activated in the presence of 5 μg/mL trypsin and incubated with 1,10 or 100 μg/mL test compound in serum-free medium at 37° C. for 30 minutes. The activated virus was then diluted 1:5 in serum-free medium containing test compound at the same concentration (to bring the trypsin concentration to 1 μg/mL), added to MA-104 cells, and serially diluted on microtiter plates (5-fold series). The diluent contained 1 μg/mL trypsin and the indicated amount of test compound. After the virus was adsorbed at 37° C. for 1 hour, the virus innoculum was removed, and the media was replaced with serum-free medium containing 1 μg/mL trypsin and the indicated amount of test compound. Cultures were incubated at 37° C. for 3 days, and then cell viability was assessed with the Promega MTT assay following manufacturer's instructions. Each plate contained a dilution series of virus not treated with test compound and each plate contained cells exposed to test compound without virus. All samples were tested in triplicate. Absorbance at 570 nm was determined on a SpectraMax microplate reader (Molecular Devices) equipped with SoftMax Pro (v 3.1.2) data analysis software. The mean absorbance of triplicate determinations, the standard deviation, and the % CV (standard deviation/mean* 100) were calculated. The Q test was used to remove outliers (Anderson, Robert L. 1987 in Practical Statistics for Analytical Chemists, page 31-33, Table 7, Appendix D). Data were fit to a 4-parameter curve using SoftMax Pro software. The curve equation is y=((A-D)/(1+(x/C)^{B}))=D which defines a sigmoid shaped curve; A is the lower asymptote as X approaches zero; D is the upper plateau as X approaches infinity; B is the slope in the linear portion of the curve; and C is the ED₅₀.

Results: Table 1 and Graph 1 show results of MA-104 cells infected with Rotavirus and treated with lactulose. Lactulose protected MA-1 04 cells from virus killing in a dose dependent manner.

Table 2 shows the results of studies comparing the effect of several oligosaccharides. Test compound and activated Rotavirus were diluted in serum free medium and added to five wells of MA-104 cells. Another 5 wells on the same microtiter plate were exposed to test compound in serum free medium containing trypsin. After three days incubation, cell viability was assessed with the MTT metabolism assay. Data are expressed as percent of control without virus or compound added.

The studied oligosaccharides were sialyllactose, kappa-carageenen, arabinogalactan, galactooligosaccharide and lactulose. Sialyllactose, arabinogalactan and galactooligosaccharide in the presence of virus resulted in significant cell kill due to virus infection (low values in MTT metabolism assay). These data are consistent with previous results that indicate these three compounds do not inhibit Rotavirus. Cells treated with virus and kappa-carageenen and lactulose remained viable. Although this finding is consistent with previous data that kappa-carageenen (U.S. Pat. No. 5,658,526) inhibits Rotavirus infection, there are no data indicating that lactulose acts in such a manner.

TABLE 1
MA-104 Infection with Lactulose (Mean OD)

| Virus | No Test | Lactulose | Lactulose | Lactulose |
|---|---|---|---|---|
| (diln)-1 | Compound | 100 μg/mL | 10 μg/mL | 1 μg/mL |
| 10 | 0.073 ± 0.002 | 0.133 ± 0.045 | 0.087 ± 0.002 | 0.072 ± |
| | | | | 0.007 |
| 50 | 0.223 ± 0.081 | 0.849 ± 0.061 | 0.315 ± 0.168 | 0.132 ± |
| | | | | 0.010 |
| 250 | 0.397 ± 0.286 | 0.906 ± 0.081 | 0.541 ± 0.330 | 0.428 ± |
| | | | | 0.092 |
| 1250 | 0.706 ± 0.030 | 0.971 ± 0.060 | 0.869 ± 0.088 | 0.756 ± |
| | | | | 0.036 |
| 6250 | 0.647 ± 0.147 | 1.012 ± 0.153 | 0.808 ± 0.085 | 0.743 ± |
| | | | | 0.064 |
| 31250 | 0.822 ± 0.038 | 0.951 ± 0.038 | 0.820 ± 0.082 | 0.782 ± |
| | | | | 0.049 |
| 156250 | 0.661 ± 0.098 | 0.942 ± 0.040 | 0.844 ± 0.030 | 0.804 ± |
| | | | | 0.031 |
| No virus | 0.454 ± 0.039 | 0.612 ± 0.192 | 0.419 ± 0.070 | 0.372 ± |
| | | | | 0.093 |

Date are mean ± sd absorbance of triplicate determinations

TABLE 2
MA-104 Cell Viability After Exposure
to Test Compound with and without Rota virus

| Test Compound | (+) Virus | (−) Virus |
|---|---|---|
| A Sialyllactose | 17% | 100% |
| B Kappa Carageenen | 119% | 112% |
| C Arabinogalactan | 19% | 102% |
| D Galactooligosaccharide | 24% | 116% |
| E Lactulose | 119% | 134% |

### EXAMPLE 2

This example illustrates an infant formula suitable for addition for use in the present invention.

TABLE 3
Nutrient Information for Infant Formula
(Enfamil ® Lipil with Iron)

| NUTRIENTS | Per 100 Calories |
|---|---|
| (Normal Dilution) | (5 fl oz) |
| Protein, g | 2.1 |
| Fat, g | 5.3 |
| Carbohydrate, g | 10.9 |
| Water, g | 134 |
| Linoleic acid, mg | 860 |
| Vitamins: | |
| A, IU | 300 |
| D, IU | 60 |
| E, IU | 2 |
| K, μg | 8 |
| Thiamin (Vitamin B1), μg | 80 |
| Riboflavin (Vitamin B2), μg | 140 |
| B6, μg | 60 |
| B12, μg | 0.3 |
| Niacin, μg | 1000 |
| Folic acid (Folacin), μg | 16 |
| Pantothenic acid, μg | 500 |
| Biotin, μg | 3 |
| C (Ascorbic acid), mg | 12 |
| Choline, mg | 12 |
| Inositol, mg | 6 |
| Minerals: | |
| Calcium, mg | 78 |
| Phosphorus, mg | 53 |
| Magnesium, mg | 8 |
| Iron, mg | 1.8 |
| Zinc, mg | 1 |
| Manganese, μg | 15 |
| Copper, μg | 75 |
| Iodine, μg | 10 |
| Selenium, μg | 2.8 |
| Sodium, mg | 27 |
| Potassium, mg | 108 |
| Chloride, mg | 63 |

The ingredients of this particular formula are: reduced minerals whey, nonfat milk, vegetable oil (palm olein, soy, coconut, and high oleic sunflower oils), lactose, and less than 1%: mortierella alpina oil, crypthecodinium cohnii oil, vitamin A palmitate, vitamin D3, vitamin E acetate, vitamin K1, thiamin hydrochloride, vitamin B6 hydrochloride, vitamin B12, niacinamide, folic acid, calcium pantothenate, biotin, sodium ascorbate, inositol, calcium chloride, calcium phosphate, ferrous sulfate, zinc sulfate, manganese sulfate, cupric sulfate, sodium chloride, sodium citrate, potassium citrate, potassium hydroxide, sodium selenite, taurine, nucleotides (adenosine 5′-monophosphate, cytidine 5′-monophosphate, disodium guanosine 5′-monophosphate, disodium uridine 5′-monophosphate).

To use this particular formula to practice the present invention, it would be necessary to add, for example, from 1.5mg to 10 mg of lactulose to the composition described in Table 3. This added amount of lactulose would be part of the total amount of carbohydrate.

### EXAMPLE 3

This example illustrates a hypoallergenic, virtually isotonic, nutritionally complete infant formula to which lactulose can be added. The formula contains medium chain triglycerides (MCT oil) as 55% of its fat blend and a protein hydrolysate, and is marketed as a powder or a ready-to-use liquid without lactulose (in units containing either 20 Calories or 24 Calories), under the name Enfamil® Pregestimil®, by Mead Johnson & Company, of Evansville, Ind.

Table 4 lists the nutrients of this particular formula. The ingredients are corn syrup solids, casein hydrolysate, medium chain triglycerides (MCT oil), dextrose, modified corn starch, soy oil, corn oil, high oleic oil (safflower or sunflower), and less than 2% vitamin A palmitate, vitamin D₃, vitamin E acetate, vitamin K₁, thiamin hydrochloride, riboflavin, vitamin B₆ hydrochloride, vitamin B₁₂, niacinamide, folic acid, calcium pantothenate, biotin, ascorbic acid, choline chloride, inositol, calcium citrate, calcium phosphate, magnesium oxide, ferrous sulfate, zinc sulfate, manganese sulfate, cupric sulfate, sodium iodide, sodium citrate, potassium citrate, potassium chloride, sodium selenite, acetylated monoglycerides, L-cystine, L-tyrosine, L-tryptophan, taurine, L-carnitine.

To use this particular formula to practice the present invention, it would be necessary to add, for example, from 1.5 mg to 10 mg of lactulose to the composition described in Table 4. This added amount of lactulose would be part of the total amount of carbohydrate.

TABLE 4
Nutrient Information for Infant Formula
(Enfamil ® Pregestimil ®)

| NUTRIENTS | | |
|---|---|---|
| (Normal Dilution) | 20 Cal | 24 Cal |
| per 100 Calories | (5 fl oz) | (4.2 fl oz) |
| Protein, g | 2.8 | 2.8 |
| Fat, g | 5.6 | 5.6 |
| Carbohydrate, g | 10.2 | 10.2 |
| Water, g | 133 | 108 |
| Linoleic acid, mg | 1040 | 1040 |
| Vitamins | | |
| Vitamin A, IU | 380 | 380 |
| Vitamin D, IU | 50 | 560 |
| Vitamin E, IU | 4 | 4 |
| Vitamin K, μg | 12 | 12 |
| Thiamin (Vitamin B₁), μg | 80 | 80 |
| Riboflavin (Vitamin B₂), μg | 90 | 90 |
| Vitamin B₆, μg | 60 | 60 |
| Vitamin B₁₂, μg | 0.3 | 0.3 |
| Niacin, μg | 1000 | 1000 |
| Folic acid (Folacin), μg | 16 | 16 |
| Pantothenic acid, μg | 500 | 500 |
| Biotin, μg | 3 | 3 |
| Vitamin C | 12 | 12 |
| (Ascorbic acid), mg | | |
| Choline, mg | 12 | 12 |
| Inositol, mg | 17 | 17 |
| Minerals | | |
| Calcium, mg | 115 | 115 |
| Phosphorus, mg | 75 | 75 |
| Magnesium, mg | 11 | 11 |
| Iron, mg | 1.8 | 1.8 |
| Zinc, mg | 1 | 1 |
| Manganese, μg | 25 | 25 |
| Copper, μg | 75 | 75 |
| Iodine, μg | 15 | 15 |
| Selenium, μg | 2.8 | 2.8 |
| Sodium, mg | 47 | 47 |
| Potassium, mg | 110 | 110 |
| Chloride, mg | 86 | 86 |

### EXAMPLE 4

This example illustrates a balanced formula for toddlers wherein lactulose can be added to practice the present invention. The formula is marketed under the name Enfamil® NextStep® by Mead Johnson & Company.

The formula contains the following ingredients: nonfat milk, corn syrup solids, vegetable oil (palm olein, soy, coconut, and high oleic sunflower oils), lactose, and less than 2% vitamin A palmitate, vitamin D₃, vitamin E acetate, vitamin K₁, thiamin hydrochloride, riboflavin, vitamin B₆ hydrochloride, vitamin B₁₂, niacinamide, folic acid, calcium pantothenate, biotin, ascorbic acid, choline chloride, inositol, calcium phosphate, ferrous sulfate, zinc sulfate, manganese sulfate, cupric sulfate, sodium selenite, taurine.

Table 5 lists the nutrients present in the formula.

TABLE 5
Nutrient Information for Infant Formula
(Enfamil ® NextStep ®)

| NUTRIENTS | Per 100 Calories |
|---|---|
| (Normal Dilution) | Powder (5 fl oz) |
| Protein, g | 2.6 |
| Fat, g | 5 |
| Carbohydrate, g | 11.1 |
| Water, g | 133 |
| Linoleic acid, mg | 810 |
| Vitamins | |
| Vitamin A, IU | 300 |
| Vitamin D, IU | 60 |
| Vitamin E, IU | 2 |
| Vitamin K, μg | 8 |
| Thiamin (Vitamin B₁), μg | 100 |
| Riboflavin (Vitamin B₂), μg | 150 |
| Vitamin B₆, μg | 60 |
| Vitamin B₁₂, μg | 0.25 |
| Niacin, μg | 1050 |
| Folic acid (Folacin), μg | 15 |
| Pantothenic acid, μg | 450 |
| Biotin, μg | 4.4 |
| Vitamin C | 9 |
| (Ascorbic acid), mg | |
| Choline, mg | 16 |
| Inositol, mg | 4.7 |
| L-carnitine, mg | 2.5 |
| Minerals | |
| Calcium, mg | 120 |
| Phosphorus, mg | 84 |
| Magnesium, mg | 8 |
| Iron, mg | 1.8 |
| Zinc, mg | 0.9 |
| Manganese, μg | 7 |
| Copper, μg | 90 |
| Iodine, μg | 8 |
| Selenium, μg | 2.8 |
| Sodium, mg | 41 |
| Potassium, mg | 130 |
| Chloride, mg | 86 |

### EXAMPLE 5

This is another example of a formula to which lactulose can be added. In this case, it is a pre-thickened formula, marketed under the name Enfamil A.R.® by Mead Johnson & Company. Enfamil A.R.® is a nutritionally complete infant formula with a calorie profile similar to routine infant formula. Its increased viscosity, however, is helpful for infants with uncomplicated gastroesophageal reflux (GER) or who otherwise want a thickened formula.

It is marketed either as a ready-to-use liquid or as a powder. The basic ingredients are: nonfat milk, vegetable oil (palm olein, soy, coconut, and high oleic sunflower oils), rice starch, lactose, maltodextrin, and less than 1% vitamin A palmitate, vitamin D₃, vitamin E acetate, vitamin K₁, thiamin hydrochloride, riboflavin, vitamin B₆ hydrochloride, vitamin B₁₂, niacin-amide, folic acid, calcium pantothenate, biotin, ascorbic acid, choline chloride, inositol, ferrous sulfate, zinc sulfate, manganese sulfate, cupric sulfate, sodium selenite, taurine.

Table 6 lists the nutrients present in the formula.

TABLE 6
Nutrient Information for Infant Formula
(Enfamil A.R. ®)

| NUTRIENTS | Per 100 Calories |
|---|---|
| (Normal Dilution) | (5 fl oz) |
| Protein, g | 2.5 |
| Fat, g | 5.1 |
| Carbohydrate, g | 11 |
| Water, g | 134 |
| Linoleic acid, mg | 860 |
| Vitamins | |
| Vitamin A, IU | 300 |
| Vitamin D, IU | 60 |
| Vitamin E, IU | 2 |
| Vitamin K, μg | 8 |
| Thiamin (Vitamin B₁), μg | 80 |
| Riboflavin (Vitamin B₂), μg | 140 |
| Vitamin B₆, μg | 60 |
| Vitamin B₁₂, μg | 0.3 |
| Niacin, μg | 1000 |
| Folic acid (Folacin), μg | 16 |
| Pantothenic acid, μg | 500 |
| Biotin, μg | 3 |
| Vitamin C | 12 |
| (Ascorbic acid), mg | |
| Choline, mg | 12 |
| Inositol, mg | 6 |
| Minerals | |
| Calcium, mg | 78 |
| Phosphorus, mg | 53 |
| Magnesium, mg | 8 |
| Iron, mg | 1.8 |
| Zinc, mg | 1 |
| Manganese, μg | 15 |
| Copper, μg | 75 |
| Iodine, μg | 10 |
| Selenium, μg | 2.8 |
| Sodium, mg | 40 |
| Potassium, mg | 108 |
| Chloride, mg | 75 |

### EXAMPLE 6

This is another example of a formula to which lactulose can be added. In this case, it is a milk-free, and sucrose-free infant formula with soy protein. It is marketed under the name Enfamil® ProSobee® by Mead Johnson & Company, as concentration liquid, ready-to-use liquid or powder. The basic ingredients for the concentrated and ready-to-use liquids are water, corn syrup solids, vegetable oil (palm olein, soy, coconut, and high oleic sunflower oils), soy protein isolate, and less than 1% soy lecithin, mono-and diglycerides, carrageenan, vitamin A palmitate, vitamin D₃, vitamin E hydrochloride, vitamin B₁₂, niacinamide, folic acid, calcium pantothenate, biotin, sodium ascorbate, choline chloride, inositol, calcium phosphate, magnesium chloride, ferrous sulfate, zinc sulfate, cupric sulfate, potassium iodide, sodium chloride, potassium citrate, potassium hydroxide, potassium chloride, sodium selenite, L-methionine, taurine, L-carnitine.

Table 7 lists the nutrients present in the formula.

TABLE 7
Nutrient Information for Infant Formula
(Enfamil ® ProSobee ®)

| NUTRIENTS | Per 100 Calories |
|---|---|
| (Normal Dilution) | (5 fl oz) |
| Protein, g | 2.5 |
| Fat, g | 5.3 |
| Carbohydrate, g | 10.6 |
| Water, g | 134 |
| Linoleic acid, mg | 860 |
| Vitamins | |
| Vitamin A, IU | 300 |
| Vitamin D, IU | 60 |
| Vitamin E, IU | 2 |
| Vitamin K, μg | 8 |
| Thiamin (Vitamin B1), μg | 80 |
| Riboflavin (Vitamin B2), μg | 90 |
| Vitamin B6, μg | 60 |
| Vitamin B12, μg | 0.3 |
| Niacin, μg | 1000 |
| Folic acid (Folacin), μg | 16 |
| Pantothenic acid, μg | 500 |
| Biotin, μg | 3 |
| Vitamin C | 12 |
| (Ascorbic acid), mg | |
| Choline, mg | 12 |
| Inositol, mg | 6 |
| Minerals | |
| Calcium, mg | 105 |
| Phosphorus, mg | 83 |
| Magnesium, mg | 11 |
| Iron, mg | 1.8 |
| Zinc, mg | 1.2 |
| Manganese, μg | 25 |
| Copper, μg | 75 |
| Iodine, μg | 15 |
| Selenium, μg | 2.8 |
| Sodium, mg | 36 |
| Potassium, mg | 120 |
| Chloride, mg | 80 |

### EXAMPLE 7

This is another example of an infant formula to which lactulose can be added. In this case, the formula is easily digestible and balanced for milk-sensitive toddlers. It is milk-free and contains high-quality soy protein, with a level of protein appropriate for toddlers. It also contains the iron that cow's milk lacks, and a complement of carbohydrates, fats, proteins, vitamins, and minerals designed to help meet the nutritional needs of growing toddlers. The formula is marketed under the name Enfamil® NextStep® Soy by Mead Johnson & Company. The basic ingredients in the formula are: Corn syrup solids, vegetable oil (palm olein, soy, coconut, and high oleic sunflower oils), soy protein isolate, sugar, and less than 2% vitamin A palmitate, vitamin D₃, vitamin E acetate, vitamin K₁, thiamin hydrochloride, riboflavin, vitamin B₆ hydrochloride, vitamin B₁₂, niacinamide, folic acid, calcium pantothenate, biotin, sodium ascorbate, choline chloride, inositol, calcium phosphate, calcium carbonate, magnesium chloride, ferrous sulfate, zinc sulfate, manganese sulfate, cupric sulfate, potassium iodide, sodium chloride, potassium citrate, potassium chloride, potassium phosphate, sodium selenite, L-methionine, taurine, L-carnitine.

Table 8 lists the nutrients present in the formula.

TABLE 8
Nutrient Information for Infant Formula
(Enfamil ® NextStep ® Soy)

| NUTRIENTS | Per 100 Calories |
|---|---|
| (Normal Dilution) | Powder (5 fl oz) |
| Protein, g | 3.3 |
| Fat, g | 4.4 |
| Carbohydrate, g | 11.8 |
| Water, g | 133 |
| Linoleic acid, mg | 72 0 |
| Vitamins | |
| Vitamin A, IU | 300 |
| Vitamin D, IU | 60 |
| Vitamin E, IU | 2 |
| Vitamin K, μg | 8 |
| Thiamin (Vitamin 81), μg | 80 |
| Riboflavin (Vitamin B2), μg | 90 |
| Vitamin B6, μg | 90 |
| Vitamin B12, μg | 0.3 |
| Niacin, μg | 1000 |
| Folic acid (Folacin), μg | 16 |
| Pantothenic acid, μg | 500 |
| Biotin, μg | 3 |
| Vitamin C (Ascorbic acid), mg | 12 |
| Choline, mg | 12 |
| Inositol, mg | 17 |
| L-carnitine, mg | 2 |
| Minerals | |
| Calcium, mg | 115 |
| Phosphorus, mg | 90 |
| Magnesium, mg | 8 |
| Iron, mg | 1.8 |
| Zinc, mg | 1.2 |
| Manganese, μg | 25 |
| Copper, μg | 75 |
| Iodine, μg | 15 |
| Selenium, μg | 2.8 |
| Sodium, mg | 48 |
| Potassium, mg | 150 |
| Chloride, mg | 100 |

### EXAMPLE 8

Lactose can be also added to multivitamin or other nutritional supplements. For example, it may be added to Poly-Vi-Sol® Drops, Poly-Vi-Sol® with iron Drops, Poly-Vi-Sol® Chewable Tablets, Poly-Vi-Sol® with Iron Chewable Tablets, four multivitamin supplements marketed by Mead Johnson & Company. The ingredients for Poly-Vi-Sol® Drops are: artificial flavor and color (caramel), ascorbic acid, ferrous sulfate (as a stabilizer for vitamin B₁₂), glycerin, niacinamide, polysorbate 80, riboflavin-5-phosphate sodium, thiamin hydrochloride, vitamin A palmitate, vitamin B6 hydrochloride, vitamin B₁₂, vitamin D₃, vitamin E succinate, water. The ingredients for Poly-Vi-Sol® with Iron Drops are: artificial flavor and color (caramel), ascorbic acid, ferrous sulfate, glycerin, niacinamide, polysorbate 80, riboflavin-5-phosphate sodium, thiamin hydrochloride, vitamin A palmitate, vitamin B₆ hydrochloride, vitamin D₃, vitamin E succinate, water. The ingredients for Poly-Vi-Sol® Chewable Tablets are artificial flavor and color (Yellow 6, Red 40, Blue 2), ascorbic acid, dextrates, folic acid, magnesium stearate, niacinamide, riboflavin, salt, silicon dioxide, sodium ascorbate, sugar, thiamin mononitrate, vitamin A acetate, vitamin B₆ hydrochloride, vitamin B₁₂, vitamin D₃, vitamin E acetate. The ingredients for Poly-Vi-Sol® with Iron Chewable Tablets are artificial flavor and color (Red 40), ascorbic acid, cupric oxide, dextrates, ferrous fumarate, folic acid, magnesium stearate, niacinamide, riboflavin, salt, silicon dioxide, sodium ascorbate, stearic acid, sugar, thiamin mononitrate, vitamin A acetate, vitamin B₆ hydrochloride, vitamin B₁₂, vitamin D₃, vitamin E acetate, zinc oxide.

Table 9 lists the vitamin contents for each these multivitamin supplements.

TABLE 9
Poly-Vi-Sol ® Multivitamin Supplement

| | Drops (per mL) | | | Tablets (per tablet) | | |
|---|---|---|---|---|---|---|
| Vi-Sol ® | | With | % | | With | % |
| Formulations | Regular | Iron | DV* | Regular | Iron | DV** |
| Vitamin A, IU | 1500 | 1500 | 100 | 2500 | 2500 | 50 |
| Vitamin D, IU | 400 | 400 | 100 | 400 | 400 | 100 |
| Vitamin E, IU | 5 | 5 | 100 | 15 | 15 | 50 |
| Vitamin C, mg | 35 | 35 | 100 | 60 | 60 | 100 |
| Folic acid, mg | — | — | — | 0.3 | 0.3 | 75 |
| Thiamin, mg | 0.5 | 0.5 | 100 | 1.05 | 1.05 | 70 |
| Riboflavin, mg | 0.6 | 0.6 | 100 | 1.2 | 1.2 | 70 |
| Niacin, mg | 8 | 8 | 100 | 13.5 | 13.5 | 68 |
| Vitamin B6, mg | 0.4 | 0.4 | 100 | 13.5 | 13.5 | 68 |
| Vitamin B12, μg | 2 | — | 100 | 4.5 | 4.5 | 75 |
| Iron, mg | — | 10± | 67 | — | 12±± | 67 |
| Copper, mg | — | — | — | — | 0.8 | 53 |
| Zinc, mg | — | — | — | — | 8 | 53 |
| Sizes | 50 | 50 | | 100's | 100's | |
| | mL | mL | | | | |

*Percentage of Daily Value (DV) for Infants.
**Percentage of Daily Value (DV) for Adults and Children 4 or more years of age.
±From 50 mg ferrous sulfate heptahydrate
±±From 40 mg ferrous fumarate

These and other modification and variations to the present invention may be practice by those of ordinary skill in the art, without departing from the spirit and scope of the present invention, which is more particularly set forth in the appended claims. In addition, it should be understood that aspect of the various embodiments might be interchanged both in whole and in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred version contained therein.

All references cited in this specification, including without limitation all papers, publications, patents, patent applications, presentations, texts, reports, manuscripts, brochures, books, internet postings, journal articles, periodicals and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

What is claimed is:

1. A method for inhibiting Rotavirus infection of animal or human cells in mammals, the method comprising the administration to the mammal of an effective Rotavirus infection-inhibiting amount of lactulose.

2. The method of claim 1 wherein lactulose is administered to children age five or under.

3. The method of claim 1 wherein lactulose is administered as part of a nutritional product.

4. The method of claim 3 wherein the nutritional product is selected from the group consisting of infant formula, follow-on formula, toddler's beverage, fruit juice, milk, yogurt, and fermented product.

5. The method of claim 3 wherein the nutritional product is infant formula.

6. The method of claim 5 wherein the infant formula is nutritionally complete.

7. The method of claim 5 wherein the infant formula comprises fats in an amount from about 3 to about 7 g/100 kcal of formula; proteins in an amount from about 1 to about 5 g/100 kcal of formula; carbohydrates in an amount from 6 to about 16 g/100 kcal of formula; vitamins and minerals.

8. The method of claim 3 wherein the nutritional product is selected from the group consisting of solid baby food, candies, cookies, and gelatines.

9. The method of claim 1 wherein lactulose is administered in liquid drops, powder, pills, capsules, tablets, lozenges, or in combination with vitamins.

10. The method of claim 1 wherein the amount of lactulose is administered from about 1.5 mg per kg of body weight to about 450 mg per kg of body weight.

11. The method of claim 10 wherein the amount of lactulose is administered from about 5 mg per kg of body weight to about 100 mg per kg of body weight.

12. A composition comprising lactulose in an amount effective to inhibit Rotavirus infection.

13. The composition comprising lactulose of claim 12 wherein the composition is a nutritional product.

14. The composition of claim 13 wherein the nutritional product is an infant formula.

15. The infant formula of claim 14 wherein the formula is for preterm infants.

16. The infant formula of claim 15 further comprising ARA and DHA.

17. The composition of claim 12 wherein the amount of lactulose is from about 1.5 mg per kg of body weight to about 450 mg per kg of body weight.

18. The composition of claim 12 wherein the amount of lactulose is from about 5 mg per kg of body weight to about 100 mg per kg of body weight.

19. The composition of claim 12 further comprising oligosaccharides.

20. The composition of claim 12 further comprising one or more live cultures of microorganisms selected from the group consisting of Lactobacillus and bifidus bacteria.

21. A composition comprising lactulose in an amount effective to ameliorate diarrhea in infants, toddlers and children.

22. The composition comprising lactulose of claim 21 wherein the composition is a nutritional product.

23. The composition of claim 22 wherein the nutritional product is an infant formula.

24. The infant formula of claim 23 wherein the formula is for preterm infants.

25. The infant formula of claim 24 further comprising ARA and DHA.

26. The composition of claim 21 wherein the amount of lactulose is from about 1.5 mg per kg of body weight to about 450 mg per kg of body weight.

27. The composition of claim 21 wherein the amount of lactulose is from about 5 mg per kg of body weight to about 100 mg per kg of body weight.

28. The composition of claim 21 further comprising oligosaccharides.

29. The composition of claim 21 comprising one or more live cultures of microorganisms selected from the group consisting of Lactobacillus and bifidus bacteria.
